(19) United States
(12) Patent Application Publication
(10) Pub. No.: US 2011/0318277 A1
(43) Pub. Date: Dec. 29, 2011

(54) TACROLIMUS COMPOSITIONS FOR AEROSOL ADMINISTRATION

(75) Inventors: Richard N. Dalby, Lutherville, MD (US); Ralph Niven, Half Moon Bay, CA (US)

(73) Assignee: APT Pharmaceuticals, Inc. University of Maryland, Baltimore

(21) Appl. No.: 13/168,856

(22) Filed: Jun. 24, 2011

Related U.S. Application Data
(60) Provisional application No. 61/358,586, filed on Jun. 25, 2010.

(51) Int. Cl. A61K 9/12; A61P 37/06

(52) U.S. Cl. 424/45

(57) ABSTRACT

The present disclosure relates generally to highly concentrated macrolide solutions for aerosol administration, and related methods of producing and administering the same. In one aspect, provided herein is a receptacle containing a highly concentrated solution of a macrolide compound such as tacrolimus, where the solution exhibits long-term stability in a pMDI canister and achieves high respirable fractions when actuated, among, other features. In yet another aspect, provided is a composition suitable for aerosol administration comprising tacrolimus dissolved in an ethanol-liquified propellant mixture, characterized by a tacrolimus concentration of greater than 0.15 weight percent (w/w) and comprising no more than about 10 weight percent (w/w) ethanol.

### CROSS-REFERENCE TO RELATED APPLICATIONS

This application claims the benefit of priority of U.S. Patent Application No. 61/358,586, filed Jun. 25, 2010, the contents of which are expressly incorporated herein by reference in their entirety.

### FIELD

The present disclosure relates generally to highly concentrated immunosuppressant macrolide solutions for aerosol administration, and related methods of producing and administering the same. More particularly, in one aspect, provided herein is a receptacle containing a highly concentrated solution of an immunosuppressive macrolide compound such as tacrolimus, where the solution exhibits long-term stability in a pMDI canister and achieves high respirable fractions when actuated, among other features.

### BACKGROUND

The macrolides represent a structure-based family of compounds characterized by a macrocyclic 12-to-16-membered lactone ring structure to which one or more sugar moieties are typically attached. The macrolides are generally structurally classified by ring size, and vary with respect to chemical substitutions on the various carbon atoms in the structure, and in their sugar moieties. One particular macrolide, tacrolimus, has been shown to be a potent immunosuppressive agent. Although their structures are completely dissimilar, the immunosuppressive properties of tacrolimus are remarkably similar to those of cyclosporine A (CsA).

Tacrolimus is a 23-membered macrolide lactone, and was originally identified in 1982 as being produced by a strain of Streptomyces isolated from a soil sample from Tsukuba, Japan. Its structure contains a hemiketal-masked α,β-diketoamide incorporated in the 23-membered lactone ring. The compound was initially identified as a potent immunosuppressant due to its specific inhibitory effects on mixed lymphocyte cultures (Ochai, et al., Transplant Proc 1987; 19:1284-6). Tacrolimus has been associated with increased success in the prevention of allograft rejection and bronchiolitis obliterans syndrome (BOS) following lung transplantation in combination with cell-cycle inhibitors such as mycophenolate and a steroid such as prednisone. Bronchiolitis obliterans syndrome is believed to be a manifestation of chronic allograft rejection and occurs in 30% to 50% of patients at two years post lung transplantation (Boehler, A., et al., Curr Opin Pulm Med., 6:133-9 (2000)). Tacrolimus is currently available as an oral formulation and sold under the tradename, PROGRAF® (Astellas Pharmaceuticals, Tokyo Japan). Tacrolimus, as a calcineurin inhibitor, has been found to be up to one hundred times more potent than the alternative immunosuppressive agent used in maintenance therapy, cyclosporine, following lung transplantation (Kino et al., J. Antibiot. (Tokyo) 40, 1256-1265 (1987).

The clinical utility of tacrolimus is hindered due to its dose related efficacy and toxicity, narrow therapeutic index, potential drug interactions, and large inter/intra patient variability in pharmacokinetics of available oral or parenteral formulations (Ihara, et al., Int. J. Urol, 2:151-5 (1995)). Due to low systemic bioavailabilities and short half-lives, high oral doses are typically required for use in solid organ transplant indications or for use in stem cell transplantation, thereby increasing the likelihood of adverse side effects and reduced patient compliance.

To overcome one or more of these problems, various oral tacrolimus formulations have been developed including those based upon complexation with dimethyl-β-cyclodextrin (Arima et al., J. Pharma Sci, 90:690-701 (2001), biodegradable microspheres (Wang et al., Am. J. of Transplantation, 4:721-6 (2004), and liposomes (Canadas, et al., Biochemistry, 43:9926-38 (2004). In the commercial product, PROGRAF®, amorphous drug is stabilized within a hydroxypropylmethylcellulose matrix. Pulmonary administration of tacrolimus has also been explored using nano-liposomes (Chougule, et al., International Journal of Nanomedicine, 2(4) 675-699 (2007) and dispersions for nebulization (Watts, et al., International Journal of Pharmaceutics, 384, 46-52 (2010). Such aerosolizable formulations and related treatments often are frequently suboptimal due to their inability to (i) stably achieve the high concentrations of drug necessary to delivery a therapeutically effective amount of tacrolimus via inhalation, (ii) form stable formulations having adequate shelf-life, and/or (iii) efficiently deliver adequate amounts of drug from the inhaler device (i.e., low emitted dose). Such formulations may also typically possess low respirable particle fractions, or for liquid dispersions, exhibit clogging or contain a high percentage of excipients for which localized delivery to the lung may be undesirable.

### SUMMARY

Provided herein are highly concentrated solution-based formulations of tacrolimus, where the tacrolimus is dissolved in mixture of a propellant combined with small quantities of a co-solvent such as ethanol, along with related methods of administration and treatment. The solution formulations possess high concentrations of tacrolimus (which remains in solution over time), and demonstrate excellent aerosol properties when administered via a metered dose inhaler.

In a first aspect, provided is a composition suitable for aerosol administration. The composition, which is a solution rather than a suspension, comprises tacrolimus dissolved in a co-solvent-liquified propellant mixture, and is characterized by a tacrolimus concentration of greater than 0.15 weight percent (% w/w) and comprises no more than about 10 weight percent (% w/w) ethanol.

In one embodiment, the composition is free of triglycerides.

In yet another embodiment, the composition is free of medium-chain triglycerides.

In yet another embodiment, the co-solvent is ethanol.

In a further embodiment, the propellant comprises a hydrofluoroalkane (HFA). In a related embodiment, the hydrofluoroalkane is selected from HFA-134a, HFA-227, and mixtures thereof.

In yet another embodiment, the composition comprises from about 1 to about 8 weight percent of ethanol. In an alternative embodiment, the composition comprises from about 2 to about 6 weight percent of ethanol.

In a particular embodiment related to any one or more of the foregoing, the concentration of tacrolimus ranges from 0.16 to about 2.5 weight percent (% w/w).

In yet a further embodiment, the composition is further characterized by an ethanol-to-HFA ratio ranging from about 1 percent (% w/w) ethanol in HFA to about 8 percent (% w/w) ethanol (or other alcoholic solvent) in HFA.

In yet another embodiment, the composition has a concentration of tacrolimus in ethanol ranging from about 50 to about 500 milligrams per milliliter.

In a further embodiment, the composition, when stored at 25° C. in a pressured metered dose inhaler (pMDI) canister, exhibits a shelf life of at least one year, where shelf life is characterized by a degree of chemical degradation of tacrolimus of no greater than 10% over a time period of one year.

In yet another embodiment, the composition, when stored at 25° C., maintains the tacrolimus in solution for a period of at least ten weeks, as determined by visual inspection. In a related embodiment, the composition when stored as described above, maintains the tacrolimus in solution for a time period of at least six months, as determined by visual inspection, or even for a time period of at least a year.

Also provided herein is any one or more of the foregoing compositions further comprising a pharmaceutically acceptable carrier or excipient.

In yet another aspect, provided herein is any one or more of the foregoing compositions in a unit dose container.

In an embodiment related to the above, the unit dose container is a propellant-based metered dose inhaler canister.

In yet another particular embodiment of the foregoing, the canister comprises tacrolimus dissolved in a liquefied propellant-ethanol mixture, where the amount of ethanol in the liquefied propellant-ethanol mixture is no more than about 8% w/w.

In yet a further embodiment, the unit dosage form comprises from about 1 to about 8 weight percent of ethanol.

In one or more particular embodiments, the unit dosage form comprises an aerosolizable composition as provided herein.

In a further embodiment, the unit dosage form, when actuated in a metered dose inhaler, exhibits a fine particle fraction (FPF) of 30% or more having a mass median aerodynamic diameter (MMAD) of <3.99 μm.

In yet another embodiment, the unit dosage form, when actuated in a metered dose inhaler, exhibits a fine particle fraction of 40% or more having a mass median aerodynamic diameter of <3.99 μm.

In another embodiment, the unit dosage form, when actuated in a metered dose inhaler, exhibits an emitted dose of greater than 90%.

In a further embodiment, the unit dosage form as provided herein, when actuated in a metered dose inhaler, provides a spray whose particles are characterized by a mass median aerodynamic diameter ranging from about 2 to about 3.5 microns, e.g., when actuated within a standardized in vitro test apparatus.

In a further aspect, provided is a method for administering a therapeutically effective amount of tacrolimus to the lungs of a subject in need thereof by aerosolized administration of a composition or unit dosage form such as described herein, e.g., at a frequency of no more than about eight times daily (e.g., from 1 to 8 times daily). In a related embodiment, aerosolized administration as described herein occurs at a frequency of no more than about 7 times daily (e.g., from 1 to 7 times daily), or no more than about 6 times daily (e.g., from about 1 to 6 times daily), or no more than about 5 times daily (e.g., from 1 to 5 times daily), or no more than about 4 times daily (e.g., from 1 to 4 times). In yet another particular embodiment, aerosolized administration as described herein occurs at a frequency of no more than about four times daily (QID).

In yet a further embodiment, the composition is administered at a dosing frequency of no more than three times daily (e.g., from 1 to 3 times daily).

In yet another embodiment, the composition is administered at a dosing frequency of no more than two times daily, or even once daily.

In yet a further aspect, provided is a method of treating or preventing lung transplant rejection in a subject, e.g., a human subject, the method comprising administering by inhalation a total daily metered dosage amount of from about 1 milligram to about 10 milligrams of tacrolimus (e.g., 1, 2, 3, 4, 5, 6, 7, 8, 9, or 10 mg), wherein the daily metered dosage amount is administered in four or fewer doses daily (e.g., one, two, three or four daily doses).

In an embodiment related to the foregoing, each of the four or fewer doses consists of a single actuation event.

In yet another embodiment, each of the four or fewer doses consists of two actuation events.

In a further aspect, provided is a method for treating a condition responsive to treatment with tacrolimus, comprising administering to the lungs of a subject suffering from such condition by inhalation of a therapeutically effective amount of a composition or unit dosage form as described herein.

In yet another aspect, the tacrolimus compositions provided herein are administered as a stand-alone therapy, i.e., in the absence of concurrent treatment with one or more additional immunosuppressants. In one such embodiment, a composition as provided herein is administered to a subject suffering from toxicity, for example, kidney toxicity, due to systemic treatment with one or more agents used to prevent transplant rejection. In yet another embodiment, a tacrolimus composition as provided herein is used as a stand-alone treatment for a post-transplant patient.

Additional embodiments of the present compositions, methods, and the like will be apparent from the following description, drawings, examples, and claims. As can be appreciated from the foregoing and following description, each and every feature described herein, and each and every combination of two or more of such features, is included within the scope of the present disclosure provided that the features included in such a combination are not mutually inconsistent. In addition, any feature or combination of features may be specifically excluded from any embodiment of the present invention. Additional aspects and advantages of the present invention are set forth in the following description and claims, particularly when considered in conjunction with the accompanying examples and drawings.

These and other objects and features of the invention will become more fully apparent when read in conjunction with the following detailed description.

### BRIEF DESCRIPTION OF THE DRAWINGS

FIG. 1 is a cross-sectional view of an exemplary metered dose inhaler (MDI) illustrating components of the device. For ease of reference, the figure demonstrates the canister or can, the headspace, the drug suspension (which may also be a solution), inhaled air entry, the crimp, the metering valve, the valve stem, the atomizing nozzle, the actuator body, and the mouthpiece.

FIG. 2 is an HPLC chromatogram of an illustrative sample of tacrolimus (obtained from a commercial source) used in the studies described herein.

FIG. 3 is a graph illustrating the chemical stability of tacrolimus in ethanol over time at different concentrations ranging from 100 to 400 mg/ml when stored at 55° C. as described in greater detail in Example 7. The plot illustrates percent degradation products over time (days).

FIG. 4 is a plot illustrating the degradant levels contained in solutions of tacrolimus dissolved in ethanol at a fixed concentration of 200 mg/ml over time (days) as a function of temperature. Details are provided in Example 8.

FIG. 5 is a plot illustrating the chemical degradation of tacrolimus contained in exemplary MDI formulations and stored in pressured MDI canisters over time under accelerated stability testing conditions. The MDI formulations of tacrolimus were prepared using different HFA propellants, HFA-134a and HFA-227 and 3% w/w/ethanol, as described in detail in Example 9.

### DETAILED DESCRIPTION

Before describing one or more embodiments of the present invention in detail, it is to be understood that this disclosure is not limited to the particular embodiments, formulations, exemplified ratios, and the like described herein, as such may vary.

The present invention now will be described more fully hereinafter. This invention may, however, be embodied in many different forms and should not be construed as limited to the embodiments set forth herein; rather, these embodiments are provided so that this disclosure will be thorough and complete, and will fully convey the scope of the invention to those skilled in the art.

All publications, patents and patent applications cited herein, whether supra or infra, are hereby incorporated by reference in their entirety.

### DEFINITIONS

In describing and claiming the present invention, the following terminology will be used in accordance with the definitions described below.

It must be noted that, as used in this specification and the intended claims, the singular forms “a,” “an,” and “the” include plural referents unless the context clearly dictates otherwise. Thus, for example, reference to “a hydrofluoroalkane” includes a single hydrofluoroalkane as well as two or more of the same or different hydrofluoroalkanes; reference to “an optional excipient” refers to a single optional excipient as well as two or more of the same or different optional excipients, and the like.

The term, tacrolimus, as well as reference to other chemical compounds herein, is meant to include the compound in any of its pharmaceutically acceptable forms, including isomers such as diastereomers and enantiomers, salts, solvates, hydrates, tautomers, polymorphs, particular crystalline forms, amorphous forms, as well as racemic mixtures and pure isomers, where applicable.

“Pharmaceutically acceptable excipient or carrier” refers to an excipient or carrier that may optionally be included in the compositions of the invention and that causes no significant adverse toxicological effects to the patient upon administration.

“Pharmaceutically acceptable salt” includes, but is not limited to, amino acid salts, salts prepared with inorganic acids, such as chloride, sulfate, phosphate, diphosphate, bromide, and nitrate salts, or salts prepared from the corresponding inorganic acid form of any of the preceding, e.g., hydrochloride, etc., or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly salts containing pharmaceutically acceptable cations include, but are not limited to, sodium, potassium, calcium, aluminum, lithium, and ammonium (including substituted ammonium).

“Emitted Dose” or “ED” provides an indication of the delivery of a drug formulation from a suitable inhaler device after a firing or dispersion event. The ED is an experimentally-determined parameter, and is typically determined using an in-vitro device set up which mimics patient dosing. For MDI and nebulizer dosage forms, the ED corresponds to the percentage of drug, e.g., tacrolimus, drawn from a dosage form and that exits the mouthpiece of an inhaler device.

A metered dose is the amount of drug delivered from a metering valve after actuation, i.e., the valve dose. The amount of drug that is delivered from the mouthpiece after actuation is the ex-actuator dose.

“Fine particle fraction” or “FPF” is defined as the mass percent of particles having an aerodynamic diameter of less than 3.99 μm, indicated by FPF_{<3.99}. The particle size analysis is determined by compendial procedures (USP 26-NF 21. Chapter 601—Physical tests and determinations: aerosols. United States Pharmacopeia. Rockville, Md.: United States Pharmacopeial Convention; 2003:2105-2123; European Pharmacopeia. Section 2.9.18—Preparations for inhalation: aerodynamic assessment of fine particles. European Pharmacopeia. 3rd ed. [Suppl 2001]. Strasbourg, France: Council of Europe; 2002:113-124). The measurement is typically undertaken using a multistage cascade impactor equipped with United States Pharmacopeia/European Pharmacopeia (USP/EP) induction port. This technique provides a direct link with the mass of therapeutically active pharmaceutical ingredient (API) and particle aerodynamic size, which is accepted as an indication of the likely deposition location within the respiratory tract (Rudolph G, Kobrich R, Stahlhofen W. Modeling and algebraic formulation of regional aerosol deposition in man. J Aerosol Sci. 1990; 21(suppl 1):306-406).

“Mass median aerodynamic diameter” or “MMAD” is a measure of the aerodynamic size of a dispersed particle. The aerodynamic diameter is used to describe an aerosolized particle in terms of its settling behavior, and is the diameter of a unit density sphere having the same settling velocity, in air, as the particle. The aerodynamic diameter encompasses particle shape, density and physical size of a particle. As used herein, MMAD refers to the midpoint or median of the aerodynamic particle size distribution of an aerosolized powder determined by inertial impaction, using for example, a New Generation Impactor or NGI available from TSI Instruments, LTD, United Kingdom. (See, e.g., Allen T. Particle size, shape, and distribution. In: Scarlett B, ed. Particle Size Measurement. 4th ed. New York, N.Y.: Chapman and Hall Inc; 1990:124-189).

“Pharmacologically effective amount” is the amount of an active agent present in an aerosolizable composition, needed to provide a desired level of active agent in the bloodstream or at the site of action (e.g., the lungs) of a subject to be treated to provide an anticipated physiological, biophysical, biochemical, or pharmacological response when such composition is administered pulmonarily. The precise amount will depend upon numerous factors, e.g., the active agent, the activity of the composition, the delivery device employed, the physical characteristics of the composition, intended patient use (i.e., the number of doses administered per day), patient considerations, and the like, and can readily be determined by one skilled in the art, based upon the information provided herein.

“Optional” or “optionally” means that the subsequently described circumstance may or may not occur, so that the description includes instances where the circumstance occurs and instances where it does not.

“Substantially absent” or “substantially free” of a certain feature or entity means nearly totally or completely absent the feature or entity. Typically, a component that is substantially free of a particular entity contains less than about 5% of that entity.

“Pharmaceutically acceptable excipient or carrier” refers to an excipient that may optionally be included in the formulations provided herein, and taken into the lungs with no significant adverse toxicological effects to the subject, and particularly to the lungs of the subject.

A tacrolimus solution that exhibits “substantially no change” in its level of tacrolimus over a given period is one that changes (i.e., degrades) by no more than ±5 percent relative to its initial level of tacrolimus. Such a composition is referred to herein as a “stabilized composition”.

The terms “subject”, “individual” or “patient” are used interchangeably herein and refer to a vertebrate, preferably a mammal. Mammals include, but are not limited to, humans, or veterinary subjects such as non-human primates, dogs, cats, horses, cows, and the like.

The terms “pharmacologically effective amount” or “therapeutically effective amount” of a tacrolimus composition as provided herein, refer to a non-toxic (or having a minimal but pharmaceutically acceptable level of toxicity) but sufficient amount of the composition or agent to provide the desired response, e.g., for improving symptoms related to a condition that is responsive to treatment with tacrolimus. The amount required will vary from subject to subject, depending on the species, age, and general condition of the subject, the severity of the condition being treated, additional drugs being taken by the subject, mode of administration, and the like. An appropriate “effective” amount in any individual case may be determined by one of ordinary skill in the art using routine experimentation, based upon the information provided herein.

“Treatment” or “treating” of a particular condition includes: (1) preventing such a condition, i.e. causing the condition not to develop, or to occur with less intensity or to a lesser degree in a subject that may be exposed to or predisposed to the condition but does not yet experience or display the condition, (2) inhibiting the condition, i.e., arresting the development or reversing the condition.

The term “about”, particularly in reference to a given quantity, is meant to encompass deviations of plus or minus five percent.

“Substantially” means nearly totally or completely, for instance, 95% or greater of some given quantity.

Additional definitions may also be found in the sections which follow.

### Overview: Tacrolimus Formulation

As described above, the present disclosure provides tacrolimus formulations suitable for aerosolized delivery, where the formulations are solutions rather than suspensions, and possess significantly higher concentrations of tacrolimus than those previously achieved. The solutions provided herein possess concentrations of tacrolimus that appear to exceed even the solubility that would be observed in pure ethanol when measured at volumes appropriate for a receptacle for use in a metered dose inhaler (MDI). Additionally, the ethanol-propellant tacrolimus solutions described herein appear to possess chemical stabilities (as measured by the extent of drug degradation over time) that are greater than in ethanol alone. Unexpectedly, the solutions provided herein exhibit greater chemical stabilities at higher concentrations of drug rather than at lower concentrations. Moreover, such features are achieved absent the addition of solubility enhancers (other than co-solvent), liposome encapsulation, surfactant addition, incorporation of stabilizers, incorporation of dispersants, and other such similar approaches which often involve the use of materials considered to be undesirable for direct delivery to lung tissue.

The solution-based formulations provided herein, based upon the unexpected solubility of tacrolimus at high concentration within the exemplary ethanol-propellant mixtures suggest that aerosol-based delivery of such formulations can be used to administer therapeutically effective doses of drug to a subject in need thereof using daily dosing regimens of no more than four doses daily, and preferably in the range of 1 to 3 doses daily. The solutions described herein, when contained in a canister suitable for use in a metered dose inhaler, provide aerosol formulations having good solution phase stability and additionally, exhibit and maintain excellent aerosol performance over time, as will be described in greater detail below.

### Formulation Components

### Tacrolimus

Tacrolimus (also FK-506 or Fujimycin) is a naturally-occurring macrolide antibiotic, and is used as an immunosuppressive drug. The chemical name for tacrolimus is 17-allyl-1,14-dihydroxy-12-[2-(4-hydroxy-3-methoxycyclohexyl)-1-methylvinyl]-23,25-dimethyoxy-13,19,21,27-tetramethyl-11,28-dioxa-4-azatricyclo-[22.3.1.0^{4,9}]octacos-18-ene-2,3,10,16-tetraone. Tacrolimus is a calcineurin inhibitor that limits the nuclear translocation of Nuclear Factor of Activated T-cells (NF-AT) which, in turn, prevents the transcription of subsequent release of inflammatory and immunostimulatory cytokines such as IL-2, and possesses a selective inhibitory effect on T-lymphocytes. The main use of tacrolimus is after allogeneic organ transplant where it suppresses the activity of the recipient's immune system, thereby lowering the risk of organ rejection. It is available in oral form (as capsules) under the brand name, PROGRAF® (Astellas Pharma, Inc.) and is also used as a topical preparation, e.g., in gel or ointment form, under the brand name, “PROTOPIC®” (Fujisawa), in the treatment of severe atopic dermatitis (“eczema”).

Tacrolimus was first described in the patent literature, e.g., in U.S. Pat. No. 4,894,366 and in European Patent No. 184,162, and later in the scientific literature (e.g., H. Tanaka et al., J. Am. Chem. Soc. 1987, 109, 5031-5033; T. Kino et al. J. Antibiot. 1987, 40, 1249-1255). Tacrolimus is typically prepared by fermentation, but its synthesis has also been described, e.g., in European Patent No. 378,318. Methods describing the separation and purification of tacrolimus can be found, e.g., in U.S. Pat. Nos. 6,492,513; 6,576,135; 6,881,341, and in International Patent Publication No. WO 05/054253.

The structure of tacrolimus is provided below:

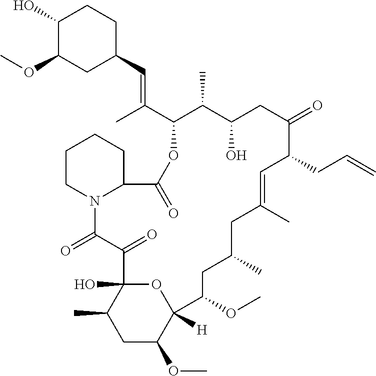

A more generalized structure, which encompasses tacrolimus and other compounds originally isolated from S. tsukubaensis, which vary in the identity of the substituent, R, is provided below.

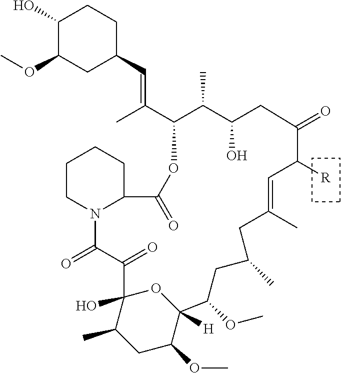

In the foregoing structure, when R is equal to allyl (—CH₂CH═CH₂), the compound corresponds to tacrolimus. Other analogs that are structurally similar to tacrolimus and encompassed by the instant disclosure include those where R=ethyl (—CH₂CH₃, ascomycin, FR900520), R=propyl (—CH₂CH₂CH₃, tsucubamycin B), and R=methyl (—CH₃, FR900523).

Tacrolimus possesses several known isomeric and tautomeric forms, where the chemical structures below indicate the region of the molecule involved in the tautomerization to form tautomers I and II. Tautomers I and II are known conformational isomers of tacrolimus, and have been previously described, e.g., in Namiki, Y. et al., “Tautomeric phenomenon of a novel potent immunosuppressant: FK506, in solution”, J. Antibiot. 46, (1993), 1149-1155. See also, Alak, A. M., Therap. Drug Monitoring, June 1992 (3), 338-351.

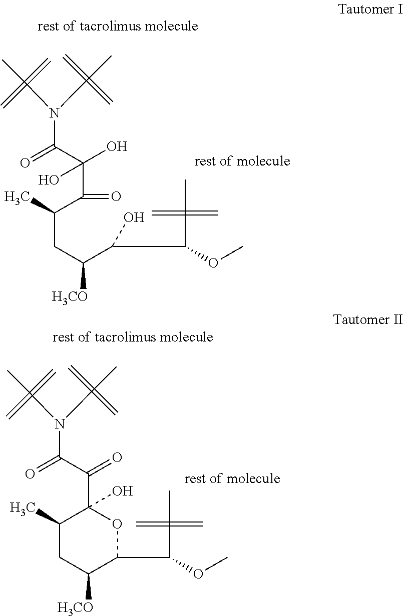

While tacrolimus is obtainable by the methods of synthesis and purification described above, tacrolimus is also obtainable from commercial sources which include the following: Haorui Pharma-Chem, Inc. (Edison, N.J.), Concord Biotech Ltd (Ahemedabad, India), Chunghwa Chemical Synthesis & Biotech Co. (Taiwan, China), and Fujian Kerui Pharmaceutical Co. (Korea). Tacrolimus for use in the instant formulations may be in any form suitable for use, i.e., it may be crystalline, amorphous, in the form of a hydrate or solvate, or the like. Purity of tacrolimus can be assessed using any suitable analytical technique such as HPLC, NMR, and the like.

As described in Example 7, commercially available tacrolimus contains only trace amounts of various impurities, in addition to trace amounts of tautomers I and II, which are identifiable by HPLC. As will be described in greater detail below, solutions of tacrolimus in an HFA liquified propellent-ethanol co-solvent mixture were found to possess unexpected solubility when evaluated at volumes suitable for use in a metered dose inhaler.

Due to its poor aqueous solubility and chemical instability, the development of inhaleable formulations and administration of therapeutically effective levels of tacrolimus to the lung has proven to be a challenge—especially in developing aerosolizable formulations absent the addition of lipophilic solubility enhancers or surfactants, which can be considered undesirable for direct administration to the lungs.

### Additional Active Agents

The formulations provided herein may optionally comprise one or more additional active agents, i.e., in addition to tacrolimus. For instance, the formulation may further comprise a corticosteroid. Representative corticosteroids include but are not limited to mometasone, ciclesonide, fluticasone, budesonide, beclomethasone, flunisolide, and triamcinolone, including pharmaceutically acceptable salt forms thereof. For example, fluticasone may be used as the propionate (or other) salt; beclomethasone may be used, e.g., in its dipropionate (or other) salt form, triamcinolone may be used in any one or more of its acetonide, benetonide, furetonide, hexacetonide, or diacetate (or other salt forms). The corticosteroid or other active agent is typically contained in the formulation in a therapeutically effective amount; such an amount can be determined by one skilled in the art, e.g., based upon current recommended dosages for inhaleable formulations containing any one or more of the foregoing corticosteroids. For example, a therapeutically effective amount of triamcinolone may range from about 200 micrograms to about 1500 micrograms administered daily (ED, meaning the amount delivered from, e.g., a metered dose inhaler). A therapeutically effective dosage of flunisolide may range, for example, from about 250 micrograms to about 5000 micrograms administered daily by inhalation. As an additional example, a therapeutically effective amount of budesonide may range from about 250 micrograms to about 1800 micrograms administered daily by inhalation. An illustrative therapeutically effective amount of fluticasone may range, e.g., from about 100 micrograms to about 400 micrograms administered daily by inhalation.

### Propellant

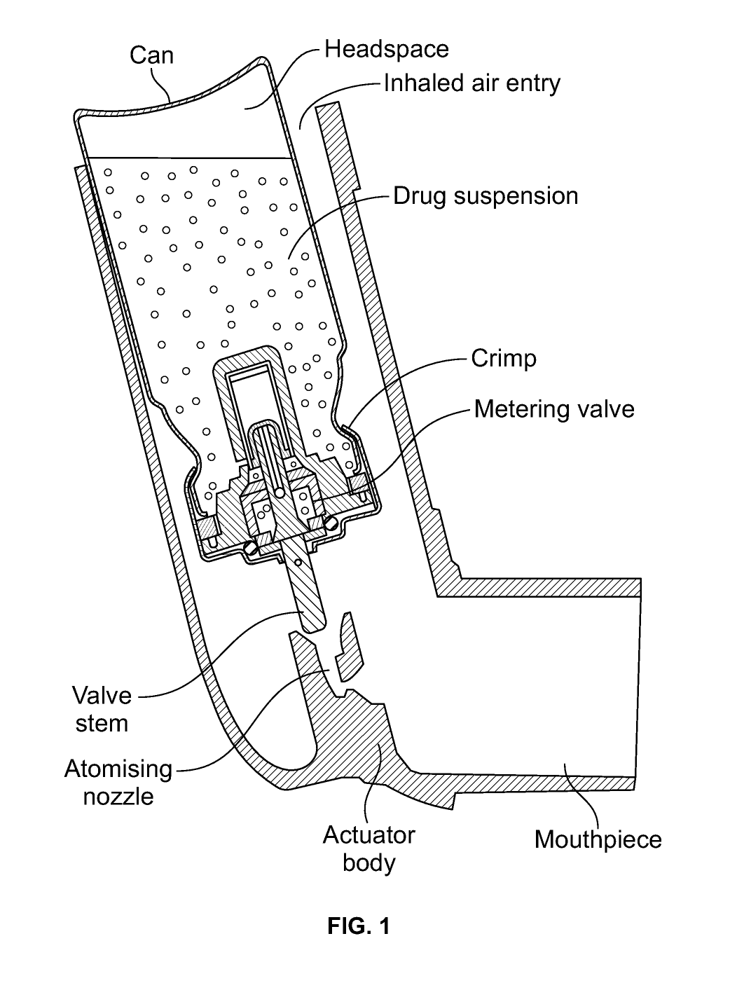

The instant solutions comprise a propellant. Propellants are used in metered dose inhalers (see, e.g., FIG. 1) to act as the storage medium and to effect aerosolization after actuation from the inhaler. After priming a metering valve within the inhaler canister, actuation results in the evacuation of the pressurized contents through a small orifice. The resulting expansion and evaporation of the propellant produces a spray or aerosol with size characteristics that are suitable for deposition in the lungs after inhalation. Propellants that may be used include liquids such as liquid chlorofluorocarbons (CFCs) or liquid fluorocarbons (also referred to as hydrofluoroalkanes or HFAs). However, due to the ozone depleting nature of chlorofluorocarbons, hydrofluoroalkanes are preferred, since these compounds are not ozone-depleting in nature. HFAs are also non-toxic and non-flammable, making certain of them appropriate for use in medical aerosols.

Illustrative CFC propellants include CCl₃F (propellant 11), CCl₂F₂ (propellant 12), C₂Cl₂F₄ (propellant 114), including mixtures thereof, although most industrialized countries have called for a ban on CFC production and use. HFAs (also referred to as HFCs) for use in the compositions described herein include 1,1,1,2-tetrafluoroethane (HFC 134a) and 1,1,1,2,3,3,3-heptafluoropropane (HFC 227ea, also referred to simply as HFC 227), preferably pharmaceutical grade, are available, for example, from Solvay Fluor. Additional exemplary hydrofluoroalkanes include, but are not limited to difluoromethane (HFC-32), 1,1,1-trifluoroethane (HFC-143(a)), 1,1,2,2-tetrafluoroethane (HFC-134), 1,1-difluoroethane (HFC-152a), as well as combinations thereof. Particularly preferred are 1,1,1,2-tetrafluoroethane (HFC-134(a)), 1,1,1,2,3,3,3-heptafluoropropane (HFC-227), and combinations thereof. An additional propellant suitable for use is dimethyl ether (DME).

Drugs such as tacrolimus, when present in therapeutically effective amounts, have minimal solubility in the HFA propellants, as do many of the pharmaceutically-acceptable excipients used in inhaleable formulations. Indeed, the lower solvency of the HFAs makes the development of solution-based aerosol formulations a significant challenge, in particular since the number of FDA-acceptable solubility enhancing agents is relatively small. As a result, many MDI formulations are suspension-based.

### Co-Solvent

The aerosolizable solutions provided herein comprise a co-solvent. “Co-solvent” as used herein refers to a substance, e.g., a liquid, in which other substances are dissolved. Co-solvents, as employed herein, are typically less volatile than propellants and are used to help dissolve a drug such as tacrolimus or other excipients in a propellant, lower the vapor pressure of the propellant system, and/or promote miscibility between propellants and immiscible solvents. Generally, a co-solvent as provided herein is an organic molecule that possesses a molecular weight of less than about 150, and a boiling point of less than about 200° C. In one embodiment, the co-solvent has a boiling point of less than about 100° C. Exemplary co-solvents include, but are not limited to alcohols, e.g. ethanol, isopropylalcohol, propanol, tert-butyl alcohol, propylene glycol, and the like. Co-solvents as provided herein are generally acceptable for pharmaceutical delivery in humans. One particular preferred co-solvent is ethanol, which is completely miscible with the hydrofluorocarbons. Although insoluble in hydrofluoroalkanes, tacrolimus is readily soluble in ethanol. While addition of a co-solvent to solubilize a drug can be advantageous (e.g., smaller particle size distributions when compared to suspension-based formulations and improved deposition in the alveolar region of the lung), the addition of a co-solvent will typically decrease the volatility of the formulation, thereby adversely impacting the efficiency of drug delivery. Thus, addition of a co-solvent, while aiding in solvency of the formulation components, can adversely impact dose consistency and reduce respirability of the formulation, thereby making the development of a formulation that delivers therapeutically effective amounts of drug while maintaining good aerosol properties a significant challenge.

### Relative Quantities

The solutions provided herein are suitable for aerosol administration and comprise tacrolimus dissolved in a co-solvent-liquified propellant mixture, e.g., an ethanol-liquified propellant mixture. The solutions are generally characterized by a tacrolimus concentration of greater than about 0.15 weight percent (% w/w) and comprise no more than about 10 weight percent (% w/w) ethanol or other suitable co-solvent. Illustrative compositions are provided in the accompanying examples. In the instances where ethanol is exemplified, it is to be understood that descriptions related thereto apply equally to any other suitable co-solvent as described herein.

As can be seen in Example 1, a composition containing 200 mg tacrolimus, 400 microliters of ethanol co-solvent and 16.7 grams of propellant HFA-134a, was filled into an aerosol bottle. Initially, a clear solution was formed that reverted to a suspension after storage overnight at room temperature. Additional formulations, all of which remained as solutions upon storage rather than forming suspensions, containing greater amounts of co-solvent are described in Examples 2-4. Results are summarized in Table 1. Particularly unexpected are the results based upon Examples 3 and 4, whereby increasing the amount of HFA in the formulation resulted in the formation of a stable solution rather than inducing precipitation as would be expected.

Generally, the preferred level of co-solvent in the formulation, e.g., ethanol or other suitable co-solvent, falls within a range of about 1-10% by mass of the propellant-ethanol mixtures. That is to say, the amount of ethanol or other co-solvent may be selected from 1%, 2%, 3%, 4%, 5%, 6%, 7%, 8%, 9%, or 10% on a weight percentage basis of the formulation. Preferably, the level of co-solvent in the formulation falls within a range of about 1-8% (% w/w). Illustrative amounts of ethanol in the solution, in percent by weight (% w/w), range from about 2-6% (% w/w) or from about 2-5% (% w/w). Ideally, a balance is achieved whereby (i) a high concentration of tacrolimus is achieved, (ii) the tacrolimus is maintained in solution, and (iii) the formulation results in good aerosolization upon device actuation.

As described above, the solutions are generally characterized by a tacrolimus concentration of greater than about 0.15 weight percent (% w/w). Typically, the concentration of tacrolimus ranges from 0.16 to about 2.5 weight percent (% w/w), although higher concentrations are envisioned. That is to say, illustrative compositions (i.e., solutions) suitably contain concentrations of tacrolimus ranging from about 0.20 to about 2.5 weight percent, or from about 0.30 to about 2 weight percent, or from about 0.40 to about 1.8 weight tacrolimus. Exemplary compositions comprise tacrolimus in an amount of about 0.20, 0.30, 0.40, 0.50, 0.60, 0.70, 0.80, 0.90, 1.0, 1.10, 1.20, 1.30, 1.40, 1.50, 1.60, 1.70, 1.80, 1.90, 2.0, 2.1, 2.2, 2.3, 2.4 or about 2.5 weight percent tacrolimus.

Illustrative ethanol-to-HFA ratios generally fall within a range of no more than about 10% w/w, and typically range from about 1 percent (% w/w) ethanol in HFA to about 8 percent (% w/w) ethanol in HFA, that is, the ratio of ethanol to HFA within the propellant. That is to say, the percent ethanol in HFA is generally selected from 1, 2, 3, 4, 5, 6, 7, and 8 percent (% w/w). Alternatively, the weight percent ethanol in HFA may fall within any one or more of the sub-ranges above.

In a particular embodiment, tacrolimus is dissolved in a liquefied propellant-ethanol mixture having a w/w ratio of ethanol-to-propellant of no more than about 8, where the tacrolimus is present at a concentration of at least 0.20 weight percent (w/w).

Additionally, the solutions provided herein will typically possess a volume ratio of ethanol to tacrolimus ranging from about 50 to about 500 milligrams per milliliter. Exemplary volume ratios are selected from about 50, 60, 70, 80, 90, 100, 150, 200, 250, 300, 350, 400, 450, and 500 milligrams per milliliter. That is to say, volume ratios of ethanol to tacrolimus may fall within a range of from 100-200, 200-300, 300-400 or 400-500 milligrams per milliliter.

### Additional Carriers and Excipients

In addition to comprising tacrolimus, a propellant and a co-solvent such as ethanol or the like, the solutions provided herein may optionally contain one or more additional pharmaceutically acceptable excipients or carriers suitable for administration to the lungs. Preferred excipients or solubility enhancers are those which are soluble in the HFA propellant, such as oligolactic acids, acyl amide acids, monofunctionalized methoxypolyethylene glycols (mPEGs) and acylated α, β, or γ-cyclodextrins (Stefely, J., Drug Development & Delivery, Vol 2 (6), September 2002).

In a particular embodiment, the solutions provided herein are absent one or more of additional stabilizers, surfactants, dispersants, solubilizers that are not co-solvents, and/or excipients. In one exemplary embodiment, the solutions/compositions provided herein are absent a triglyceride.

Illustrative pharmaceutical excipients along with other excipients are described in “Remington: The Science & Practice of Pharmacy”, 19^{th} ed., Williams & Williams, (1995), the “Physician's Desk Reference”, 52^{nd} ed., Medical Economics, Montvale, N.J. (1998), and Kibbe, A. H., Handbook of Pharmaceutical Excipients, 3^{rd} Edition, American Pharmaceutical Association, Washington, D.C., 2000.

### Administration

The solutions provided herein are suitable for administration via a metered dose inhalers or MDI as shown in FIG. 1. MDIs comprise a pressure resistant container or canister (“can”) typically filled with an active agent such as tacrolimus dissolved or suspended in a liquefied propellant, a mixture of propellants, or a mixture of solvents, propellants, and/or other excipients in compact pressurized aerosol dispensers. The pressure resistant container is fitted with a metering valve and an actuator. Actuation of the metering valve aerosolizes and releases a measured dose of the active agent, i.e., tacrolimus. The drug is typically delivered to a subject via inhalation of the aerosol. When actuated, the liquefied propellant emerges through the valve stem and actuation or atomising nozzle, expands and vaporizes resulting in aerosolization of the dissolved drug particles for delivery to the lungs of a subject during the subject's inhalation maneuver. An MDI may discharge up to several hundred metered doses of drug, depending upon the volume of the canister and other formulation-related features. Furthermore, the dose volume actuated will depend on the size of the metering valve. Typical valve sizes are 50 μl and 63 μl but larger valve sizes are available and have been used. Exemplary solution formulations suitable for use in a MDI possess a weight-to-volume ratio of tacrolimus-to-ethanol ranging from about 50 to 500 mg/ml, and a level of co-solvent in the formulation (% w/w) ranging from about 1 to 8% w/w of the propellant-co-solvent mixture. Representative formulations (ethanol-to-HFA canister ratios) based upon different dosing regimens are provided in Example 4.

FIG. 1 illustrates the components of a typical pressurized metered dose inhaler. Such components include the canister (which can be made out of glass, stainless steel, aluminum or alloys and which can be coated or uncoated); the drug solution or suspension retained within a propellant mixture that may or may not include a co-solvent such as ethanol; a metering valve assembly that is mechanically crimped onto the can and the actuator body (which often incorporates the mouth piece and atomizing nozzle). The spray characteristics are dictated by the nature of the formulation, the metering valve size, the stem and nozzle characteristics.

The solution formulations provided herein possess many advantages over suspension type formulations which may include one or more of the following: fewer issues surrounding homogenous valve sampling, enhanced efficiency of aerosolization and high degree of lung deposition, lack of particle growth issues such as with suspension-based formulations, no drug deposition on valve components and canister, simpler canister filling, among others.

### Aerosol Features

Illustrative aerosol features of the solutions described herein upon actuation from a metered dose inhaler are provided in Example 6. Formulations as provided herein, when delivered via a metered dose inhaler, typically exhibit emitted dose (ED) values of 90% or higher. That is to say, exemplary solution formulations possess ED values of at least 90%, typically on the order of at least 91%, 92%, 93%, 94%, 95%, 96%, 97%, 98%, or even 99% or higher. See Table 2. Moreover, the solution formulations maintain their aerosol performance over time. For example, preferred solutions will maintain an ED of 90% or higher when stored at room temperature over a period of 20 days. Preferably, the solutions maintain an ED of 90% or higher when stored at room temperature over a period of 30 days, or 60 days, or 90 days, one year, or longer.

Similarly, the solutions provided herein exhibit high fine particle fractions or FPF values which, similar to ED values, are maintained over time—illustrating the excellent aerosol performance of the instant formulations. Generally, a solution as provided herein, when actuated in a metered dose inhaler, exhibits a fine particle fraction of 30% or more, and more preferably, of 40% or more. Illustrative FPF values include greater than 30%, or greater than 35%, or greater than 40%, or greater than 45%, or even greater than 50%.

Further, with respect to aerosol performance, the instant solutions, when actuated in a metered dose inhaler, provide a spray whose particles are characterized by a mass median aerodynamic diameter (MMAD) ranging from about 2 to about 5 microns, or from about 2 to about 3.5 microns, preferably from about 2 to about 3 microns when measured using an appropriate sizing instrument and test apparatus such as a New Generation Impactor™ or NGI™ (TSI Instruments, LTD, United Kingdom). Illustrative MMAD values are selected from 2, 2.5, 3.0, 3.5, 4.0, 4.5 and 5.0 microns. Average MMAD values for the illustrative solutions described herein maintained values from about 2.3 to about 2.6 microns over a time period of 21 days. The particle sizes observed are ideally suited for deposition in the small airways and alveoli.

### Storage Stability/Shelf Life

Generally, the solutions provided herein possess extended shelf lives. In one embodiment, a solution as described herein, when stored at 25° C. in a pMDI canister, exhibits a shelf life of at least one year, where shelf life is characterized by a degree of chemical degradation of tacrolimus of no greater than 10% during the at least one year, measured at the one year time point. Ideally, the extent of chemical degradation of the solution under such storage conditions is no greater than 9%, or 8%, or 7% or 6% or even 5% at the one year time point.

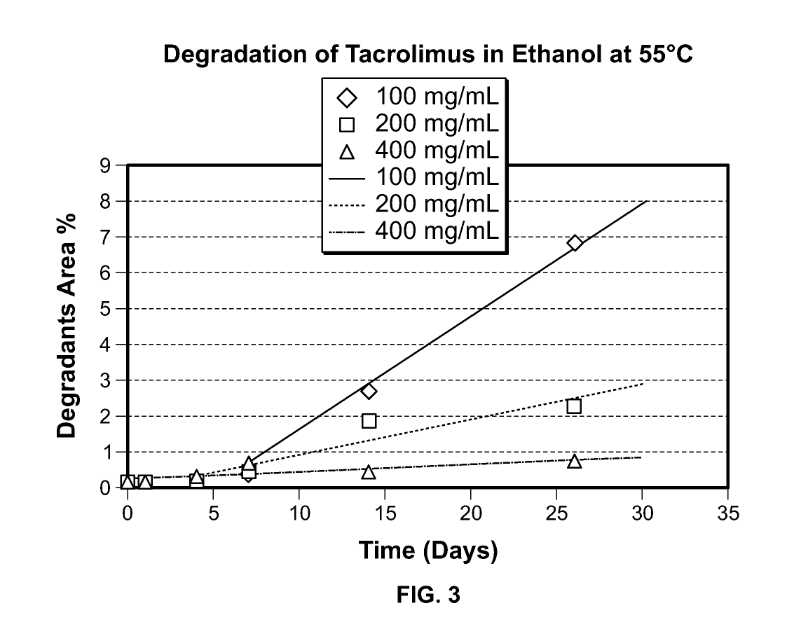

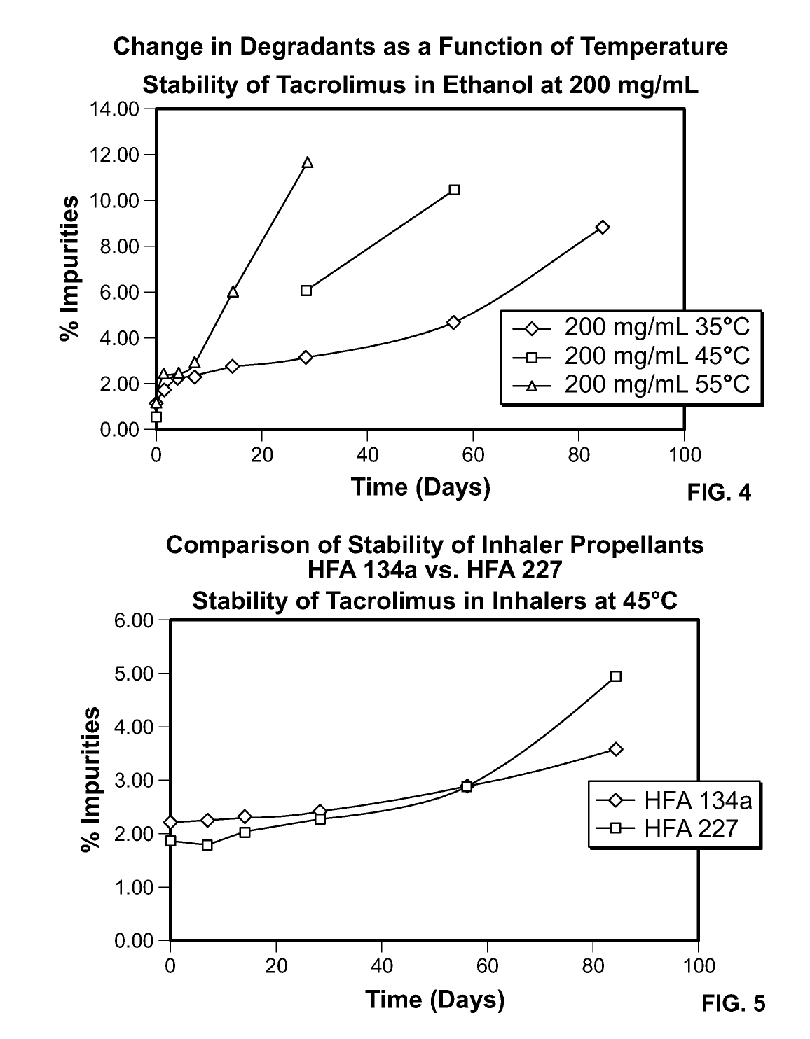

Such chemical stability is illustrated in Examples 7-9, where both a low rate and low degree of chemical degradation over time is described. First, the chemical stability of ethanol-tacrolimus solutions was determined at various concentrations of tacrolimus under accelerated storage conditions (55° C.). Based upon the results described in Example 7, the solution having the highest concentration of tacrolimus, 400 mg/ml, surprisingly exhibited the greatest stability. Based upon these results, the solution phase stability of tacrolimus in ethanol, in this case, appears to be an inverse function of concentration. Results are shown graphically in FIG. 3. The chemical stability of tacrolimus as a function of temperature was also investigated. See Example 8. Results are provided in FIG. 4, and demonstrate that, as might be expected, the rate of degradation of tacrolimus increases at higher temperatures. Finally, stability studies were carried out on MDI formulations of tacrolimus utilizing different HFAs (Example 9). Results are summarized in Table 5. Unexpectedly, it appears that the stability of tacrolimus in HFA-based formulations is greater than it is in ethanol alone, which is surprising based upon the solubility of tacrolimus in each of co-solvent and HFA.

### Treatment

The tacrolimus formulations provided herein are useful in treating a number of conditions for which tacrolimus has been shown to be effective. For example, tacrolimus may be administered to a subject suffering from one of more of the following conditions including the prevention and treatment of acute and chronic transplant rejection such as following a lung or other organ transplant (Griffin, B. P., et al., Transplantation, 1994 Mar. 27; 57(6):848-51), bronchiolitis obliterans, pulmonary sarcoidosis, chronic obstructive pulmonary disease, interstitial lung disease, idiopathic pulmonary fibrosis, adult respiratory distress syndrome, bronchiectasis, lung eosinophilia, interstitial fibrosis, cystic fibrosis, asthma, emphysema, and inflammatory lung injury. Studies in rats have shown the viability of delivery of tacrolimus to the systemic circulation via inhalation. See, e.g., Ide, N., et al., Thorac Cardiovasc Surg 2007; 133:548-553; Deuse, T., et al., Am. J. Respir. Cell Mol. Biol., 2009. Peters, J., et al., Chest, Meeting, 27 Oct. 2008.

A subject that may be suffering from possible transplant rejection may exhibit one or more of the following symptoms: fever over 100° C., flu-like symptoms such as chills, aches, headache, dizziness, and nausea, chest congestion, cough, shortness of breath, new pain or tenderness surrounding the transplanted organ, and or fatigue to name a few. Generally, transplant rejection such as lung transplant rejection can by identified by carrying out one or more of the following tests: blood work (e.g., complete blood count, bronchoscopy, and lung biopsy. Treatment or prevention of rejection may be carried out using the compositions and methods described herein.

In a particular embodiment, the formulations provided herein are effective in prolonging allograft survival. See, e.g., Vincenti, F., et al., Transplantation, 2002, 73, (5), 775-782, which describes the efficacy of tacrolimus in prolonging allograft survival. In another embodiment the formulations provided herein are effective in preventing graft vs host disease after, for example, stem cell transplantation. See, e.g., Ratanatharathorn, V., at aL, Blood, 1998, 92 (7), 2303-2314; Yamakazi, R., et al., Biology of Blood and Marrow Transplantation, 12(2) Supp. 1; 2006, 69.

Subjects to whom tacrolimus may be administered include both children (aged three months to 18 years) and adults (18 years and older).

A therapeutic amount of tacrolimus can be empirically determined and will vary with the particular condition being treated, the subject, and the like. The actual dose to be administered will vary depending upon the age, weight, and general condition of the subject as well as the severity of the condition being treated, the judgment of the health care professional, and particular dosage form being administered.

A therapeutically effective amount of tacrolimus can be determined by those skilled in the art, and will be adjusted to the requirements of each particular case. Generally, a therapeutically effective amount of tacrolimus for an adult will range from a total daily metered dosage of about 1 mg/day to about 10 mg/day, or from about 2 mg/day to about 10 mg/day, or from about 2 mg/day to about 8 mg/day, or from about 3 mg/day to about 5 mg/day, administered as either a single dosage or as multiple daily dosages, typically from one to four doses. An exemplary therapeutically effective daily dosage of tacrolimus is, e.g., selected from the following in mg/day: 1, 2, 3, 4, 5, 6, 7, 8, 9, and 10. Preferred in certain embodiments are divided dosages over the course of a day, e.g., a recommended daily dose divided into five doses, or four doses, or three doses, or two doses, or even a single dose. Preferably, the desired daily dosage is administered in 3 or fewer doses during the day, or as two or fewer doses during the day. In one particular embodiment, the target regimen is two puffs (per dose), two times per day to achieve the recommended daily dose. Alternatively, the regimen consists of two puffs per dose, once daily, or one puff per dose, once daily (i.e., a single administration event). The amount actually delivered to and deposited in the lungs preferably lies in the range of about 300 micrograms to about 900 micrograms daily (e.g., about 300 mg, 350 mg, 400 mg, 450 mg, 500 mg, 550 mg, 600 mg, 650 mg, 700 mg, 750 mg, 800 mg, 850 mg, or about 900 mg daily). In a particular embodiment, the amount of tacrolimus delivered to and deposited in the lungs is about 600 micrograms daily.

The compositions provided herein may, in one aspect, be used as stand-alone therapy (i.e., in the absence of administration of one or more additional immunosuppressants) for treating any one or more of the conditions described herein. For instance, a tacrolimus composition as provided may be used to treat a subject suffering from toxicity such as kidney toxicity, due to systemic treatment with one or more immunosuppressants; alternatively, such therapy may be used for treatment of any post-transplant patient to suppress/prevent organ rejection.

Depending upon the dosage amount and precise condition to be treated, administration can be over the course of several days, weeks, months, and even years, and may even be for the life of the patient. Illustrative dosing regimes will last a period of at least about a day, a week, from about 1-4 weeks, from 1-3 months, from 1-6 months, from 1-50 weeks, from 1-12 months, or longer.

Practically speaking, a unit dose of any given tacrolimus composition can be administered in a variety of dosing schedules, depending on the judgment of the clinician, needs of the patient, and so forth. The specific dosing schedule will be known by those of ordinary skill in the art or can be determined using routine methods.

It is to be understood that while the invention has been described in conjunction with the preferred specific embodiments thereof, that the foregoing description as well as the examples that follow are intended to illustrate and not limit the scope of the invention. Other aspects, advantages and modifications within the scope of the invention will be apparent to those skilled in the art to which the invention pertains.

All articles, books, patents and other publications referenced herein are hereby incorporated by reference in their entireties.

### EXAMPLES

### Materials

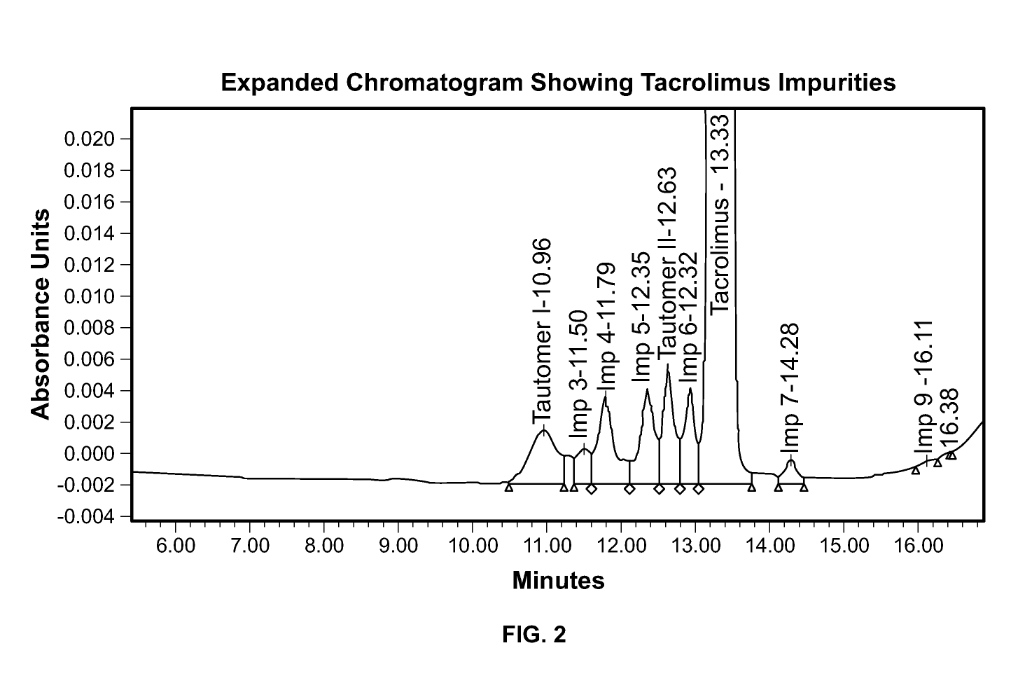

Tacrolimus powder was obtained from various commercial sources and determined to possess a purity ranging from 95% to 99.5% by HPLC (wavelength, 205 nm, 220 nm). Material was obtained from LC Laboratories, Haorui Pharma-Chem. Inc., Chunghwa Chemical Synthesis & Biotech. Co., and Fujian Kerui Pharmaceutical Co. See FIG. 2.

HFA-134a was obtained from DuPont Chemicals (Wilmington, Del.).

HFA-227 was obtained from Solvay Fluorides Inc. (Frankfurt, GE).

### Example 1

### HFA-Ethanol Tacrolimus (Precipitated) Formulation

The following example describes the preparation of a HFA-134a: ethanol formulation containing a high concentration of tacrolimus. The active agent, tacrolimus, precipitated due to inadequate quantities of co-solvent in the formulation. (Tacrolimus is readily soluble in ethanol but not in hydrofluoroalkanes; indeed tacrolimus is essentially insoluble in the hydrofluoroalkanes).

An accurate quantity of 200 mg tacrolimus powder was weighed into an individual plastic-coated glass aerosol bottle prior to the addition of 400 μl of ethanol using a volumetric pipette. Continuous valves were immediately crimped onto the aerosol bottle and approximately 16.7 g of HFA-134a was filled incrementally into the bottle using a pressure burette. The final concentration of tacrolimus in ethanol approached 400 mg/ml and the final concentration in the mixture was approximately 11.7 mg/g.

A clear solution was initially formed after shaking and sonication. The clear solution reverted to a suspension upon overnight storage at room temperature, indicating that the initial solution was supersaturated. After an additional 10 weeks of storage, the suspension evolved, generating large, transparent crystals that were visible by eye.

### Example 2

### Highly Concentrated, Solution-Stable HFA-Ethanol Tacrolimus Formulation Using HFA-134A

An accurate quantity of 200 mg tacrolimus powder was weighed into an individual plastic-coated glass aerosol bottle prior to the addition of 750 μl of ethanol using a volumetric pipette. A continuous valve was immediately crimped onto the aerosol bottle and ˜16.6 g of HFA-134a was filled incrementally into the bottle using a pressure burette. The final concentration of tacrolimus in ethanol approached 267 mg/ml, and the final concentration in the mixture was approximately 11.5 mg/g.

A clear solution was initially formed after shaking and sonication that remained as a solution after overnight storage at room temperature, and subsequently at −20° C. and −78° C. Upon warming to room temperature, the solution unexpectedly remained as a solution over ten weeks of observation and storage.

### Example 3

### Highly Concentrated, Solution-Stable HFA-Ethanol Tacrolimus Formulations Using HFA-227

An accurate quantity of 200 mg tacrolimus powder was weighed into an individual plastic-coated glass aerosol bottle prior to the addition of 1000 μl of ethanol using a volumetric pipette. A continuous valve was immediately crimped onto the aerosol bottle and ˜7.8 g of HFA-227 was filled incrementally into the bottle using a pressure burette. The final concentration of tacrolimus in ethanol was ˜200 mg/ml and the final concentration in the mixture was approximately ˜25.6 mg/g.

As in the previous example, the sample remained as a solution for at least 10 weeks of storage at room temperature and was also subjected to storage at −78° C., where it also surprisingly remained as a solution, even at this low temperature.

An equivalent formulation containing ˜22.9 g of HFA-227 behaved in a similar manner. This suggests that these formulations can be filled using cold-filling as well as well as pressure-filling techniques.

### Example 4

### Storage Stability of a Highly Concentrated, HFA-227:Ethanol Tacrolimus Solution Formulation

An accurate quantity of 200 mg tacrolimus powder was weighed into an individual plastic-coated glass aerosol bottle prior to the addition of 750 μl of ethanol using a volumetric pipette. A continuous valve was immediately crimped onto the aerosol bottle and ˜16.6 g of HFA-227 was filled incrementally into the bottle using a pressure burette. The final concentration of tacrolimus in ethanol was ˜267 mg/ml and the final concentration in the mixture was approximately ˜11.5 mg/g.

The formulation was stored at various temperatures from room temperature down to −78° C. and remained in solution under all conditions observed.

Table 1 provides a summary of the quantities of hydrofluoroalkane, co-solvent (EtOH), and drug (tacrolimus) investigated in Examples 1-4.

TABLE 1

| | | | | Tacrolimus | | | EtOH |
|---|---|---|---|---|---|---|---|
| | | Tacrolimus | EtOH, | concn in EtOH | HFA | tac/mixture | ratio |
| Sample | Ppt? | (mg) | (μl) [mg] | (mg/ml) | (g) | (w/wₜₒₜ %) | w/wₜₒₜ % |
| Ex. 1 | Yes | 200 | 400 [310] | 500 | 16.7 | 1.16 | 1.8 |
| Ex. 2 | No | 200 | 750 [590] | 267 | 16.6 | 1.15 | 3.4 |
| Ex. 3 | No | 200 | 1000 [789] | 200 | 7.8 | 2.20 | 9.0 |
| Ex. 4 | No | 200 | 1000 [789] | 200 | 22.9 | 0.84 | 3.3 |

The high concentrations of tacrolimus achieved in the MDI canisters absent precipitation were totally unexpected. Ethanol is completely miscible with the HFA, but the drug is essentially insoluble in the hydrofluorocarbons. Consequently, adding HFA to the ethanolic solutions of tacrolimus at the high concentrations employed was expected to result in precipitation. However, in many instances, such as in Examples 2-4, this was observed not to be the case. In contrast to expectations, an apparently stable solution was formed that did not precipitate even when held at temperatures as low as −70° C.

Preferred levels of co-solvent, i.e., ethanol or other alcoholic solvents, in formulations of tacrolimus suitable for use in a metered dose inhaler, relative to both drug and overall formulation will typically fall within the ranges of 1-10% by mass of the HFA-ethanol mixtures. For practical purposes, the upper limit of ethanol in HFAs is approximately 8% by mass. Higher fractions of ethanol tends to result in aerosol sprays that are unsuitable for inhalation purposes when actuated from a metered dose inhaler (MDI). More preferred is a range of ethanol of 2-6% by mass, and even more preferred is 2-5% by mass. Ideally, the ethanol (or other suitable alcohol) concentration should be kept low enough to allow good aerosol formation on actuation from the MDI but also high enough to maintain the tacrolimus in solution. The range of tacrolimus concentrations in ethanol that can be used to deliver tacrolimus in amounts anticipated to be acceptable for treatment or prophylaxis of conditions such as acute or chronic lung transplant rejection is from about 50 mg/ml to about 400 mg/ml. Generally, on a weight percent basis, the amount of tacrolimus in a formulation as provided herein and suitable for use in a MDI is no less than about 0.15 weight percent, and is typically more than about 0.15 weight percent. See, e.g., the exemplary weight percentages of tacrolimus provided in Table 1. Formulations suitable for use in a metered dose inhaler will typically possess an equivalent concentration of tacrolimus in ethanol ranging from about 50 to about 500 mg/ml, and a weight percentage of ethanol in the overall formulation (w/w %) ranging from about 1 to 8.

### Example 5

### Illustrative HFA:Ethanol Tacrolimus Solution Formulations Suitable for Inhalation Therapy

The following is a formulation scenario based on a target of 600 μg lung deposition from a metered dose inhaler. Assuming a 25% lung deposition efficiency, a 50 μl metered valve and a 10% actuator loss, the total daily metered dose requirement is 2667 μg drug.

Based upon the foregoing, the ethanol-HFA canister % w/w ratios are summarized in Table 2 below.

TABLE 2

| | ETHANOL % | ETHANOL % | ETHANOL % |
|---|---|---|---|
| | IN HFA | IN HFA | IN HFA |
| TACROLIMUS | [w/w %] 2 | [w/w %] 2 | [w/w %] 2 |
| CONC | ACTUATIONS | ACTUATIONS | ACTUATIONS |
| (MG/ML) | DAILY | BID | QID |
| 50 | 26.8 | 19.2 | 9.1 |
| 100 | 19.2 | 9.1 | 4.5 |
| 200 | 9.1 | 4.5 | 2.2 |
| 300 | 6 | 3 | 1.5 |
| 400 | 4.5 | 2.2 | 1.1 |
| 500 | 3.6 | 1.8 | 0.9 |

For the 50 μl valve volume, a lower limit of ethanol concentration that can be used is about 50 mg/ml. That is to say, preferred formulations possess an ethanol concentration of greater than about 50 mg/ml at this valve volume. The use of a 63 μl metered valve, however, would generate an ethanol weight percentage of 7.2%, based on a dosing regimen of 2 actuations 4 times daily. Such a regimen is reasonable for daily use with current inhalation therapies, but higher frequency dose regimens are not unfeasible for certain disease states. Table 2 therefore highlights the required ethanol in propellant concentrations (% w/w) for a hypothetical number of dosing regimens and tacrolimus concentrations that would be necessary to achieve a daily metering dose of 2667 ug. As can be seen from the Table, based upon the dosing regimen, particulars related to the metered dose inhaler employed and number of actuations, only some of the dosing regimens in conjunction with tacrolimus would be acceptable as aerosolizable formulations based upon the desire to achieve an ethanol concentration of less than 8%.

### Example 6

### Short Term Aerosol Performance Stability for an HFA-2270Ethanol Tacrolimus Formulation

The HFA-227 formulation containing 3% w/w ethanol and 95.2 mg tacrolimus was prepared for performance testing as follows. The bottles containing HFA227-A (200 mg tacrolimus and 750 μL EtOH) was chilled in a dry ice and methanol bath and the valve removed prior to adding the formulation to a pre-chilled, 17 ml uncoated aluminum container. The container was crimped with a 63 μL metering valve to permit particle sizing by cascade impaction.

### Initial Spray Characterization

Cascade impaction was conducted using a Next Generation Pharmaceutical Impactor™ (NGI™). No pre-separator was used. The NGI™ was operated according to the manufacturer's instructions and data was analyzed using the methods described in United States Pharmacopeia 30. This consisted of assembly of the NGI with all impaction surfaces/collection cups in place and surrounded by intact sealing o-rings. The USP induction port (sometimes known as the “throat”) was connected to NGI inlet and a regulated vacuum pump was connected to the outlet nipple. Prior to each experiment, the airflow rate was confirmed to be 30 L/min using a mass flow meter connected to the inlet of the induction port. Airflow through the NGI was continued while the flow meter was removed and the pMDI actuator mouthpiece was connected to the induction port using Parafilm®.

The experiments were conducted in an air conditioned building but humidity and temperature were not specifically controlled. After priming three times, each of the two test pressurized inhalers was separately actuated 5 times into the NGI with shaking between each actuation. Thirty seconds after the last actuation, the airflow was stopped and the apparatus disassembled. The wash volume used to extract each NGI cup and the induction port was 10 ml. The actuator wash volume was 20 ml. The extraction solvent was 50% ACN in water. All samples and standards were injected in duplicate and the peak results averaged.

A summary of aerosol performance of the formulation over time is provided in the table below.

TABLE 3
Summary of Aerosol Performance Over Time.

| | Day | | | | | |
|---|---|---|---|---|---|---|
| Particle Sizing Parameters | 0 | 1 | 2 | 14 | 16 | 21 |
| Formulation | Average | Average | Average | Average | Average | Average |
| MASS BALANCE (μg) | 476.60 | 533.41 | 497.85 | 331.47 | 370.15 | 435.15 |
| EMITTED DOSE (μg) | 431.48 | 499.55 | 465.27 | 313.10 | 362.20 | 413.62 |
| Emitted % | 90.53 | 93.65 | 93.46 | 94.46 | 97.85 | 95.05 |
| FINE PARTICAL DOSE | 196.83 | 183.85 | 211.65 | 134.98 | 106.60 | 133.96 |
| (<3.99 μm (μg)) | | | | | | |
| FINE PARTICAL FRACTION | 45.56 | 36.82 | 45.50 | 43.00 | 30.61 | 32.39 |
| (<3.99 um (%)) | | | | | | |
| MMAD (μm) | 2.40 | 2.35 | 2.44 | 2.58 | 2.39 | 2.34 |
| GSD (sigma g) | 1.97 | 1.94 | 1.91 | 1.92 | 1.58 | 1.72 |

As can be seen from the results in the table above, the formulation maintained excellent aerosol performance over time. The percent emitted dose (% ED) ranged from an average high value of approximately 98% (day 16) to an average low value of 91% (day 0). Thus, over the time period of the study, the formulation maintained excellent ED values that remained higher than 90%. The formulation similarly exhibited fine particle fraction (FPF) values that remained on average greater than 30%, with an FPF value on day 0 of approximately 46% (i.e., 46% of the particles were sized less than 3.99 μm). These formulation results were not optimized and routine FPFs>40% are anticipated with optimized formulations and testing equipment. Finally, the average MMAD (mass median aerodynamic diameter of the emitted particles) remained constant over time, ranging from approximately 2.3 to 2.6 microns. Such sizes are ideally suited for deposition in the small airways and alveoli.

Thus, the aerosol formulations provided herein not only exhibit good solution phase stability at high concentrations of tacrolimus, but also exhibit and maintain good aerosol performance over time. Moreover, the high respirable fraction exhibited by the formulations is much higher than the respirable fractions associated with typical suspension products. The combination of good solution phase stability at the drug concentrations described, coupled with good aerosol performance are features that are highly desirable for an inhalable drug product. Further, based upon the high tacrolimus concentrations stably-achieved, daily dosing at a frequency not to exceed four times a day (or QID) is achievable.

### Example 7

### Chemical Stability of Tacrolimus in Ethanol as a Function of Concentration

The chemical stability of tacrolimus in ethanol at different concentrations ranging from 100 to 400 mg/ml when stored at 55° C. was investigated.

Samples were analyzed using reverse phase HPLC with an acetonitrile-water gradient and UV detection. Samples were prepared in a non-aqueous medium, i.e., acetonitrile, to limit the interconversion of tautomers and to promote stability. Column temperature was set at 60° C. to further promote equilibrium between the cis-trans isomers. The gradient was set at 5% acetronitrile with a run time of 20 minutes. Acetic acid (0.002%) was added to mobile phase A (water). A solution of tacrolimus in 1:1 acetonitrile-water was incubated at 55° C. for 45 minutes to obtain an equilibrium mixture of the 3 tautomers so that retention times of each tautomer could be obtained.

TABLE 4
HPLC Conditions

| | |
|---|---|
| ANALYTICAL COLUMN | Waters Symmetry, C18, 4.6 × |
| | 150 mm, 3.5 μm |
| MOBILE PHASE | A: 0.002% acetic acid in water |
| | B: acetonitrile |
| FLOW RATE | 1.0 mL/min |
| GRADIENT | 65% A to 5% A over 15 minutes, then |
| | back to 65% A for 5.5 minutes |
| DETECTION | 205-220 nm |
| COLUMN TEMPERATURE | 60° C. |
| SAMPLE COMPARTMENT | 15° C. |
| INJECTION VOLUME | 25 μL |
| RUN TIME | 20 minutes |
| SAMPLE PREPARATION | 0.5 mg/mL in acetonitrile |

As a baseline for comparison, an HPLC chromatogram of tacrolimus as obtained from the vendor is provided in FIG. 2. As can be seen from FIG. 2, commercially available tacrolimus contains trace amounts of various impurities as well as two tautomeric forms indicated as tautomer I and tautomer II in the chromatogram.

Results of the chemical stability study are provided in FIG. 3. The degradation of tacrolimus in ethanol over time at 55° C. was measured for solutions at concentrations of 100 mg/ml, 200 mg/ml and 400 mg/ml. As can be seen, the solution exhibiting the greatest degree of solution-phase stability of tacrolimus over time was the solution having the highest concentration of tacrolimus at 400 mg/ml. At 26 days, this solution exhibited less than 1% degradation of tacrolimus, and also possessed the slowest rate of degradation relative to the two less concentrated solutions, while surprisingly, the solution having the lowest concentration of tacrolimus in ethanol, at 100 mg/ml, showed the greatest rate and extent of degradation at 26 days, approximately 7%.

Based upon these preliminary results, it appears, unexpectedly, that the more highly concentrated solutions of tacrolimus may demonstrate greater stabilities.

### Example 8

### Chemical Stability of Tacrolimus as a Function of Temperature

The degradant levels contained in solutions of tacrolimus dissolved in ethanol at a fixed concentration of 200 mg/ml were determined over time as a function of temperature. Samples were stored at the following temperatures: 35° C., 45° C., and 55° C.

The results are shown in FIG. 4, where percentage impurities for each storage temperature are plotted over time (to a total of 100 days). As expected, the rate of degradation of tacrolimus increased at higher temperatures. For example, at around day 30, the solution stored at 55° C. contained an impurity level of about 11%, while the solution stored at 35° C. contained only about 3% impurities.

### Example 9

### Accelerated Stability Study: MDI formulations of Tacrolimus

MDI formulations of tacrolimus were prepared using 3% w/w ethanol concentrations in different HFA propellants, HFA-134a and HFA-227. The resulting solution formulations were filled into pressured pMDI canisters and stored under accelerated stability conditions at 45° C. At various time points (Days 0, 7, 15, 1 month, 2 months, and 3 months), the samples were analyzed to determine degradant levels of tacrolimus.

Results are shown in FIG. 5 and summarized in Table 5 below. The results indicate that the formulations at room temperature are expected to exhibit a greater than 1 year stability shelf life at a temperature of 25° C. Based upon these results, the HFA-134a based formulation appears to have a somewhat better chemical stability over time relative to the HFA-227 based formulation.

At the initiation of the study, equilibration between the tacrolimus tautomers had already taken place. In general, the stability of tacrolimus at elevated temperatures within the ethanol-HFA mixtures within the pressurized canisters is better than within ethanol as shown in the Table. (200 GH refers to the stability of two samples of tacrolimus in ethanol held at the same temperature). Thus, it unexpectedly appears that the stability of tacrolimus in HFA-based formulations is greater than it is in ethanol alone.

TABLE 5

| | Concentration | | | % Purity by HPLC | | | | | |
|---|---|---|---|---|---|---|---|---|---|
| Sample | (mg/mL) | | Temp | Day | Day | Day | 1 | 2 | 3 |
| Name | Target | Actual | (° C.) | 0 | 7 | 15 | Month | Months | Months |
| A134a | 1 | — | 45 | 97.78 | 97.74 | 97.71 | 97.56 | 97.08 | 96.36 |
| | mg/spray | | | | | | | | |
| A227 | 1 | — | 45 | 98.16 | 98.21 | 97.97 | 97.73 | 97.08 | 95.01 |
| | mg/spray | | | | | | | | |
| 200GH | 200 | 218.1 | 45 | 99.34 | — | — | 93.93 | 89.49 | |

### Example 10

### Results of Aerosol Exposure Studies of Tacrolimus in Rats

A total of 54 male Sprague-dawley rats of approximately 250 g body weight were exposed to aerosols of tacrolimus (in a propylene-glycol:ethanol mixture) generated by jet-nebulizer for 30 minutes. Actual aerosol concentrations were 0.037 and 0.37 mg/L respectively for the starting solutions of 50 and 150 mg/ml, respectively. Particle size and size distributions were determined to be 2.53 (1.71) and 2.56 (1.73) μm MMAD (GSD) for the 50 and 150 mg/ml solutions, respectively. Estimated deposited doses for the 30 minute exposure (assuming 10% deposition efficiency) were 0.072 and 0.256 mg/kg for the “low” and “high” dose groups. Lung and blood samples were obtained for groups of animals (n=3/time point).

The resulting lung and blood pharmacokinetic data are illustrated in the following tables and illustrate, as expected, increasing lung and blood levels with increasing dose administered. The data also illustrate that (a) tacrolimus can be aerosolized by nebulizer, and (b) high levels of tacrolimus in the lung are achieved by aerosol administration, thus pointing to the advantages of localized administration of therapeutically effective levels of tacrolimus to the lung via aerosol-based administration.

TABLE 6
Estimate of Dose per Exposure Group

| | | | | Aerosol or | Est. |
|---|---|---|---|---|---|
| | | | | Solution | Deposited |
| Exposure | Route of | Ave | RMV | Concentration | Dose |
| Group | Admin. | BW (g) | (LPM) | (mg/L) | (mg/kg) |
| TA | IV | 250 | — | 0.800 | 0.400 |
| TB | IH | 250 | 0.163 | 0.037 | 0.072 |
| TC | IH | 250 | 0.163 | 0.136 | 0.256 |

IH = inhalation

TABLE 7
Pharmacokinetic Parameters
Blood Data

| | Dose | Cmax | Tmax | t_{1/2} | AUCₗₐₛₜ |
|---|---|---|---|---|---|
| Study Group | (mg/kg) | (ng/mL) | (hr) | (hr) | (hr · ng/mL) |
| Low | 0.072 | 40.01 | 0.083 | 2.9871 | 121 |
| Inhalation | | | | | |
| High | 0.256 | 148.64 | 0.083 | 1.7409 | 194 |
| Inhalation | | | | | |
| IV Bolus | 0.400 | 78.16 | 0.083 | 1.9024 | 179 |

TABLE 8
Lung Data

| | Dose | Cmax | Tmax | t_{1/2} | AUCₗₐₛₜ |
|---|---|---|---|---|---|
| Study Group | (mg/kg) | (ng/g) | (hr) | (hr) | (hr · ng/g) |
| Low | 0.072 | 9956.51 | 0.083 | 9.1398 | 19722 |
| Inhalation | | | | | |
| High | 0.256 | 18114.18 | 0.083 | 23.4008 | 22750 |
| Inhalation | | | | | |
| IV Bolus | 0.400 | 19081.8 | 0.083 | 24.4727 | 334095 |

1. A triglyceride-free composition suitable for aerosol administration comprising tacrolimus dissolved in a co-solvent liquefied propellant mixture, characterized by a tacrolimus concentration of greater than 0.15 weight percent (w/w) and comprising from about 2 to about 10 weight percent (w/w) ethanol.

2. The composition of claim 1, wherein the concentration of tacrolimus ranges from about 0.20 to about 2.5 weight percent (% w/w).

3. The composition of claim 1, wherein the propellant comprises a hydrofluoroalkane (HFA).

4. The composition of claim 1, comprising from about 2 to about 6 weight percent ethanol.

5. The composition of claim 1, wherein the concentration of tacrolimus ranges from 0.16 to about 2.5 weight percent (% w/w).

6. The composition of claim 3, further characterized by an ethanol-to-HFA ratio ranging from about 2 percent (% w/w) ethanol in HFA to about 8 percent (% w/w) ethanol in HFA.

7. The composition of claim 2, having a concentration of tacrolimus in ethanol ranging from about 50 to about 500 milligrams per milliliter.

8. The composition of claim 1, which when stored at 25° C. in a pressurized metered dose inhaler (pMDI) canister, exhibits a shelf life of at least one year, where shelf life is characterized by a degree of chemical degradation of tacrolimus of no greater than 10% over a time period of one year.

9. The composition of claim 3, wherein the HFA is selected from HFA-134a, HFA-227, and mixtures thereof.

10. The composition of claim 1, which, when stored at 25° C., maintains the tacrolimus in solution for a period of at least ten weeks, as determined by visual inspection.

11. The composition of claim 1 in a unit dose container.

12-20. (canceled)
